(19) United States
(12) Patent Application Publication
(10) Pub. No.: US 2015/0165412 A1
(43) Pub. Date: Jun. 18, 2015

(54) METHANE CONVERSION APPARATUS AND PROCESS USING A SUPERSONIC FLOW REACTOR

(71) Applicant: UOP LLC, Des Plaines, IL (US)

(72) Inventors: Debarshi Majumder, Forest Park, IL (US); Laura E. Leonard, Western Springs, IL (US)

(73) Assignee: UOP LLC, Des Plaines, IL (US)

(21) Appl. No.: 14/104,927

(22) Filed: Dec. 12, 2013

(51) Int. Cl. B01J 19/10; C07C 5/35

(52) U.S. Cl. CPC B01J 19/10; C07C 5/35

(57) ABSTRACT

Apparatus and methods are provided for converting methane in a feed stream to acetylene. A hydrocarbon stream is introduced into a supersonic reactor and pyrolyzed to convert at least a portion of the methane to acetylene. The reactor effluent stream may be treated to convert acetylene to another hydrocarbon process. An acid washing system is employed to wash the reactor effluent to remove any copper acetylide byproducts that may be present in the reactor effluent, or alternatively to decompose any copper acetylide byproducts that may remain in the reactor after shutdown of the reactor.

### TECHNICAL FIELD

Apparatus and methods are disclosed for converting methane in a hydrocarbon stream to acetylene using a supersonic flow reactor.

### BACKGROUND

Light olefin materials, including ethylene and propylene, represent a large portion of the worldwide demand in the petrochemical industry. Light olefins are used in the production of numerous chemical products via polymerization, oligomerization, alkylation, and other well-known chemical reactions. These light olefins are essential building blocks for the modern petrochemical and chemical industries. Producing large quantities of light olefin material in an economical manner, therefore, is a focus in the petrochemical industry. The main source for these materials in present day refining is the steam cracking of petroleum feeds.

The cracking of hydrocarbons brought about by heating a feedstock material in a furnace has long been used to produce useful products, including for example, olefin products. For example, ethylene, which is among the more important products in the chemical industry, can be produced by the pyrolysis of feedstocks ranging from light paraffins, such as ethane and propane, to heavier fractions such as naphtha. Typically, the lighter feedstocks produce higher ethylene yields (50-55% for ethane compared to 25-30% for naphtha); however, the cost of the feedstock is more likely to determine which is used. Historically, naphtha cracking has provided the largest source of ethylene, followed by ethane and propane pyrolysis, cracking, or dehydrogenation. Due to the large demand for ethylene and other light olefinic materials, however, the cost of these traditional feeds has steadily increased.

Energy consumption is another cost factor impacting the pyrolytic production of chemical products from various feedstocks. Over the past several decades, there have been significant improvements in the efficiency of the pyrolysis process that have reduced the costs of production. In a typical or conventional pyrolysis plant, a feedstock passes through a plurality of heat exchanger tubes where it is heated externally to a pyrolysis temperature by the combustion products of fuel oil or natural gas and air. One of the more important steps taken to minimize production costs has been the reduction of the residence time for a feedstock in the heat exchanger tubes of a pyrolysis furnace. Reduction of the residence time increases the yield of the desired product while reducing the production of heavier by-products that tend to foul the pyrolysis tube walls. However, there is little room left to improve the residence times or overall energy consumption in traditional pyrolysis processes.

More recent attempts to decrease light olefin production costs include utilizing alternative processes and/or feed streams. In one approach, hydrocarbon oxygenates and more specifically methanol or dimethylether (DME) are used as an alternative feedstock for producing light olefin products. Oxygenates can be produced from available materials such as coal, natural gas, recycled plastics, various carbon waste streams from industry and various products and by-products from the agricultural industry. Making methanol and other oxygenates from these types of raw materials is well established and typically includes one or more generally known processes such as the manufacture of synthesis gas using a nickel or cobalt catalyst in a steam reforming step followed by a methanol synthesis step at relatively high pressure using a copper-based catalyst.

Once the oxygenates are formed, the process includes catalytically converting the oxygenates, such as methanol, into the desired light olefin products in an oxygenate to olefin (OTO) process. Techniques for converting oxygenates, such as methanol to light olefins (MTO), are described in U.S. Pat. No. 4,387,263, which discloses a process that utilizes a catalytic conversion zone containing a zeolitic type catalyst. U.S. Pat. No. 4,587,373 discloses using a zeolitic catalyst like ZSM-5 for purposes of making light olefins. U.S. Pat. No. 5,095,163; U.S. Pat. No. 5,126,308; and U.S. Pat. No. 5,191,141 on the other hand, disclose an MTO conversion technology utilizing a non-zeolitic molecular sieve catalytic material, such as a metal aluminophosphate (ELAPO) molecular sieve. OTO and MTO processes, while useful, utilize an indirect process for forming a desired hydrocarbon product by first converting a feed to an oxygenate and subsequently converting the oxygenate to the hydrocarbon product. This indirect route of production is often associated with energy and cost penalties, often reducing the advantage gained by using a less expensive feed material.

Recently, attempts have been made to use pyrolysis to convert natural gas to ethylene. U.S. Pat. No. 7,183,451 discloses heating natural gas to a temperature at which a fraction is converted to hydrogen and a hydrocarbon product such as acetylene or ethylene. The product stream is then quenched to stop further reaction and subsequently reacted in the presence of a catalyst to form liquids to be transported. The liquids ultimately produced include naphtha, gasoline, or diesel. While this method may be effective for converting a portion of natural gas to acetylene or ethylene, it is estimated that this approach will provide only about a 40% yield of acetylene from a methane feed stream. While it has been identified that higher temperatures in conjunction with short residence times can increase the yield, technical limitations prevent further improvement to this process in this regard.

While the foregoing traditional pyrolysis systems provide solutions for converting ethane and propane into other useful hydrocarbon products, they have proven either ineffective or uneconomical for converting methane into these other products, such as, for example ethylene. While methane-to-olefin technology is promising, these processes can be expensive due to the indirect approach of forming the desired product. Due to continued increases in the price of feeds for traditional processes, such as ethane and naphtha, and the abundant supply and corresponding low cost of natural gas and other methane sources available, for example the more recent accessibility of shale gas, it is desirable to provide commercially feasible and cost effective ways to use methane as a feed for producing ethylene and other useful hydrocarbons.

Recently, the inventors herein have disclosed a supersonic reactor that employs short residence times. See co-pending and commonly-assigned U.S. patent application Ser. No. 13/967,334, “METHANE CONVERSION APPARATUS AND PROCESS USING A SUPERSONIC FLOW REACTOR,” filed Aug. 14, 2013, the contents of which are incorporated herein by reference in their entirety. A copper or copper-alloy-based metallurgy with external cooling is a preferred material for the wall of the supersonic reactor to handle the significantly higher temperature (>1500 ° C.) in the pyrolysis zone compared to traditional methane pyrolysis. The use of copper in the presence of acetylene (product of pyrolysis), however, undesirably results in formation of copper acetylides, part of which is carried out in the reactor effluent due to the high momentum of supersonic flow in the reactor. The agglomeration of copper acetylides in a downstream unit, which is characterized by lower turbulence, presents a significant risk of violent explosion and rupture of the downstream unit.

Accordingly, it is desirable to provide improved an improved methane to olefins conversion process. Further, it is desirable to provide an methane to olefin conversion process that is configured to eliminate the presence of copper acetylides in processing units downstream from the methane to olefin reactor to reduce the chance of an explosion. Furthermore, other desirable features and characteristics of the present disclosure will become apparent from the subsequent detailed description and the appended claims, taken in conjunction with the accompanying drawings and this background.

### BRIEF SUMMARY

Apparatus and methods are disclosed for converting methane in a hydrocarbon stream to acetylene using a supersonic flow reactor. In one embodiment, a system for producing acetylene from a feed stream comprising methane includes a supersonic reactor for receiving the methane feed stream and heating the methane feed stream to a pyrolysis temperature; a reactor shell of the supersonic reactor for defining a reactor chamber; a combustion zone of the supersonic reactor for combusting a fuel source to provide a high temperature carrier gas passing through the reactor space at supersonic speeds to heat and accelerate the methane feed stream to a pyrolysis temperature; at least a portion of the reactor shell comprises at least one of copper and a copper alloy, wherein upon heating and accelerating the methane stream, a reactor effluent is generated comprising acetylene and a copper acetylide byproduct; and an acid washing unit that washes the reactor effluent from the supersonic reactor with an acid to decompose the copper acetylide byproduct.

In another embodiment, a method for producing acetylene from a feed stream comprising methane includes receiving the methane feed stream and heating the methane feed stream to a pyrolysis temperature in a pyrolysis reactor, wherein at least a portion of the pyrolysis reactor comprises at least one of copper and a copper alloy; combusting a fuel source to provide a high temperature carrier gas passing through the reactor space at supersonic speeds to heat and accelerate the methane feed stream to a pyrolysis temperature, wherein upon heating and accelerating the methane stream, a reactor effluent is generated comprising acetylene and a copper acetylide byproduct; and washing the reactor effluent from the supersonic reactor in an acid wash to decompose the copper acetylide byproduct.

In yet another embodiment, a system for producing acetylene from a feed stream including methane includes a supersonic reactor for receiving the methane feed stream and heating the methane feed stream to a pyrolysis temperature, a reactor shell of the supersonic reactor for defining a reactor chamber, a combustion zone of the supersonic reactor for combusting a fuel source to provide a high temperature carrier gas passing through the reactor space at supersonic speeds to heat and accelerate the methane feed stream to a pyrolysis temperature, and at least a portion of the reactor shell comprises at least one of copper and a copper alloy. Upon heating and accelerating the methane stream, a reactor byproduct is generated within the combustion zone comprising copper acetylide. The system further includes an acid washing unit that washes at least a portion of the supersonic reactor with an acid after shutdown of the supersonic reactor to decompose any copper acetylide byproduct that may remain in the supersonic reactor after shutdown.

### BRIEF DESCRIPTION OF THE DRAWINGS

FIG. 1 is a side cross-sectional view of a supersonic reactor in accordance with various embodiments described herein; and

FIG. 2 is a schematic view of a system for converting methane into acetylene and other hydrocarbon products employing a copper acetylide removal system in accordance with various embodiments described herein.

### DETAILED DESCRIPTION

The following detailed description is merely exemplary in nature and is not intended to limit the application and uses of the embodiments described. Furthermore, there is no intention to be bound by any theory presented in the preceding background or the following detailed description.

Embodiments of the present disclosure are generally directed to apparatus and methods for converting methane in a hydrocarbon stream to acetylene using a supersonic flow reactor, while minimizing the possibility of explosion due to the presence of copper acetylides. The methane pyrolysis reactor zone presents a strong reducing environment in the presence of hydrogen and high temperature. Cu or CuO is found to interact readily with acetylene leading to copper acetylide formation. Due to the high momentum of flow in the reactor, at least a portion of the copper acetylide formed is carried in the reactor effluent, even when methods to prevent the formation of acetylides or in-situ removal procedures are used. The presently described embodiments employ a system to remove copper acetylide from the reactor effluent. The system uses an acid wash of the reactor effluent, which decomposes the acetylides. The acid wash section can be designed before or after the optional soot removal system or quench tower, and is followed by an acid neutralization unit or acid recovery separator system.

Prior to the discussion of copper acetylide decomposition and removal, a description of an exemplary supersonic flow reactor is provided. Of course, it will be appreciated that other embodiments thereof will be suitable for use with the presently described embodiments. As used herein, the term “methane feed stream” includes any feed stream comprising methane. The methane feed streams provided for processing in the supersonic reactor generally include methane and form at least a portion of a process stream. The apparatus and methods presented herein convert at least a portion of the methane to a desired product hydrocarbon compound to produce a product stream having a higher concentration of the product hydrocarbon compound relative to the feed stream.

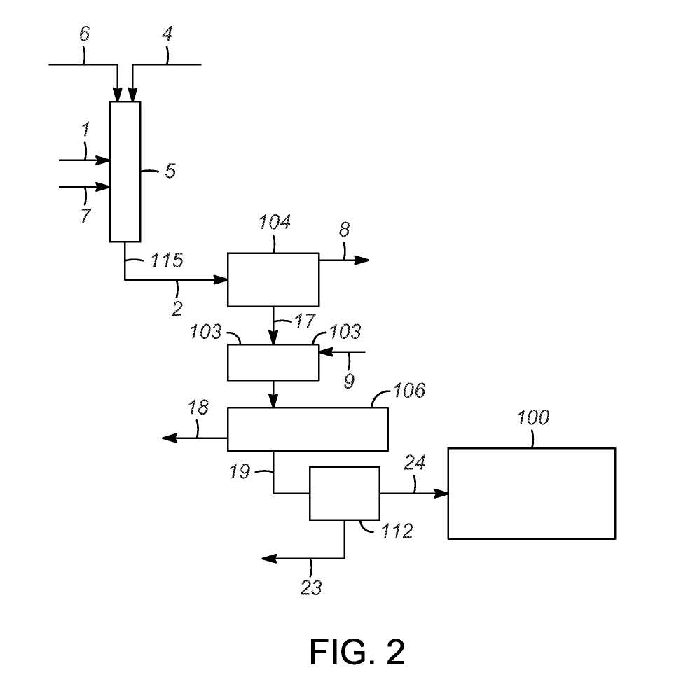

The term “hydrocarbon stream” as used herein refers to one or more streams that provide at least a portion of the methane feed stream entering the supersonic reactor as described herein or are produced from the supersonic reactor from the methane feed stream, regardless of whether further treatment or processing is conducted on such hydrocarbon stream. With reference to the example illustrated in FIG. 2, showing a conversion system 10, the “hydrocarbon stream” may include the methane feed stream 1 or a supersonic reactor effluent stream 2, or any intermediate or by-product streams formed during the processes described herein. The hydrocarbon stream may be carried via a process stream line 115, as shown in FIG. 2, which includes lines for carrying each of the portions of the process stream described above. The term “process stream” as used herein includes the “hydrocarbon stream” as described above, as well as it may include a quench fluid stream 7 (such as water or oil), a fuel stream 4 (such as hydrogen, methane, natural gas, or any suitable combustible stream), an oxygen source stream 6 (such as air, oxygen, or combinations thereof), or any streams used in the systems and the processes described herein. The process stream may be carried via a process stream line 115, which includes lines for carrying each of the portions of the process stream described above. As illustrated in FIG. 2, any of methane feed stream 1, fuel stream 4, quench fluid stream 7, and oxygen source stream 6, may be preheated, for example, by one or more heaters (not illustrated).

In accordance with various embodiments disclosed herein, therefore, apparatus and methods for converting methane in hydrocarbon streams to acetylene and other products are provided. Apparatus in accordance herewith, and the use thereof, have been identified to improve the overall process for the pyrolysis of light alkane feeds, including methane feeds, to acetylene and other useful products. The apparatus and processes described herein also improve the ability of the apparatus and associated components and equipment thereof to withstand degradation and possible failure due to extreme operating conditions within the reactor.

In accordance with one approach, the apparatus and methods disclosed herein are used to treat a hydrocarbon process stream to convert at least a portion of methane in the hydrocarbon process stream to acetylene. The hydrocarbon process stream described herein includes the methane feed stream provided to the system, which includes methane and may also include ethane or propane. The methane feed stream may also include combinations of methane, ethane, and propane at various concentrations and may also include other hydrocarbon compounds as well as contaminants. In one approach, the hydrocarbon feed stream includes natural gas. The natural gas may be provided from a variety of sources including, but not limited to, gas fields, oil fields, coal fields, fracking of shale fields, biomass, and landfill gas. In another approach, the methane feed stream can include a stream from another portion of a refinery or processing plant. For example, light alkanes, including methane, are often separated during processing of crude oil into various products and a methane feed stream may be provided from one of these sources. These streams may be provided from the same refinery or different refinery or from a refinery off gas. The methane feed stream may include a stream from combinations of different sources as well.

In one example, the methane feed stream has a methane content ranging from about 65 mol-% to about 100 mol-%. In another example, the concentration of methane in the hydrocarbon feed ranges from about 80 mol-% to about 100 mol-% of the hydrocarbon feed. In yet another example, the concentration of methane ranges from about 90 mol-% to about 100 mol-% of the hydrocarbon feed.

In one example, the concentration of ethane in the methane feed ranges from about 0 mol-% to about 35 mol-% and in another example from about 0 mol-% to about 10 mol-%. In one example, the concentration of propane in the methane feed ranges from about 0 mol-% to about 5 mol-% and in another example from about 0 mol-% to about 1 mol-%.

The methane feed stream may also include heavy hydrocarbons, such as aromatics, paraffinic, olefinic, and naphthenic hydrocarbons. These heavy hydrocarbons if present will likely be present at concentrations of between about 0 mol-% and about 100 mol-%. In another example, they may be present at concentrations of between about 0 mol-% and 10 mol-% and may be present at between about 0 mol-% and 2 mol-%.

The apparatus and method for forming acetylene from the methane feed stream described herein utilizes a supersonic flow reactor for pyrolyzing methane in the feed stream to form acetylene. The supersonic flow reactor may include one or more reactors capable of creating a supersonic flow of a carrier fluid and the methane feed stream and expanding the carrier fluid to initiate the pyrolysis reaction. In one approach, the process may include a supersonic reactor as generally described in U.S. Pat. No. 4,724,272, which is incorporated herein by reference, in its entirety. In another approach, the process and system may include a supersonic reactor such as described as a “shock wave” reactor in U.S. Pat. No. 5,219,530 and U.S. Pat. No. 5,300,216, which are incorporated herein by reference, in their entirety. In yet another approach, the supersonic reactor described as a “shock wave” reactor may include a reactor such as described in “Supersonic Injection and Mixing in the Shock Wave Reactor” Robert G. Cerff, University of Washington Graduate School, 2010.

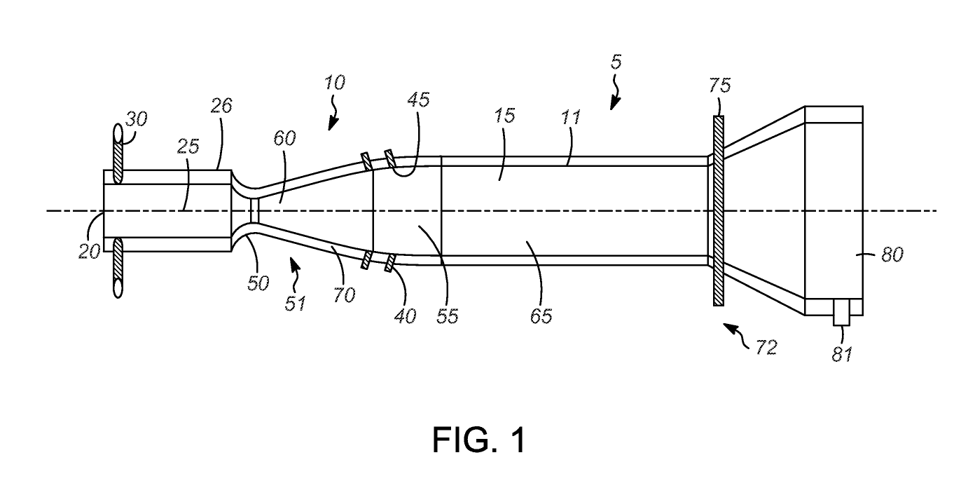

While a variety of supersonic reactors may be used in the present process, an exemplary reactor 5 is illustrated in FIG. 1. Referring to FIG. 1, the supersonic reactor 5 includes a reactor vessel 10 generally defining a reactor chamber 15. While the reactor 5 is illustrated as a single reactor, it should be understood that it may be formed modularly or as separate vessels. If formed modularly or as separate components, the modules or separate components of the reactor may be joined together permanently or temporarily, or may be separate from one another with fluids contained by other means, such as, for example, differential pressure adjustment between them. A combustion zone or chamber 25 is provided for combusting a fuel to produce a carrier fluid with the desired temperature and flowrate. The reactor 5 may optionally include a carrier fluid inlet 20 for introducing a supplemental carrier fluid into the reactor. One or more fuel injectors 30 are provided for injecting a combustible fuel, for example hydrogen, into the combustion chamber 26. The same or other injectors may be provided for injecting an oxygen source into the combustion chamber 26 to facilitate combustion of the fuel. The fuel and oxygen source injection may be in an axial direction, tangential direction, radial direction, or other direction, including a combination of directions. The fuel and oxygen are combusted to produce a hot carrier fluid stream typically having a temperature of from about 1200 to about 3500° C. in one example, between about 2000 and about 3500° C. in another example, and between about 2500 and about 3200° C. in yet another example. It is also contemplated herein to produce the hot carrier fluid stream by other known methods, including non-combustion methods. According to one example the carrier fluid stream has a pressure of about 1 atm or higher, greater than about 2 atm in another example, and greater than about 4 atm in another example.

The hot carrier fluid stream from the combustion zone 25 is passed through a supersonic expander 51 that includes a converging-diverging nozzle 50 to accelerate the velocity of the carrier fluid to above about mach 1.0 in one example, between about mach 1.0 and mach 4.0 in another example, and between about mach 1.5 and 3.5 in another example. In this regard, the residence time of the fluid in the reactor portion of the supersonic flow reactor is less than about 100 ms in one example, about 50 ms in another example, and about 10 ms in another example. The temperature of the carrier fluid stream through the supersonic expander by one example is between about 1000° C. and about 3500° C., between about 1200° C. and about 2500° C. in another example, and between about 1200° C. and about 2000° C. in another example.

A feedstock inlet 40 is provided for injecting the methane feed stream into the reactor 5 to mix with the carrier fluid. The feedstock inlet 40 may include one or more injectors 45 for injecting the feedstock into the nozzle 50, a mixing zone 55, or a reaction zone or chamber 65. The injector 45 may include a manifold, including for example a plurality of injection ports or nozzles for injecting the feed into the reactor 5.

In one approach, the reactor 5 may include a mixing zone 55 for mixing of the carrier fluid and the feed stream. In one approach, as illustrated in FIG. 1, the reactor 5 may have a separate mixing zone, between for example the supersonic expander 51 and the reaction zone 65, while in another approach, the mixing zone is integrated into the reaction zone 65, and mixing may occur in the nozzle 50, expansion zone 60, or reaction zone 65 of the reactor 5. An expansion zone 60 includes a diverging wall 70 to produce a rapid reduction in the velocity of the gases flowing therethrough, to convert the kinetic energy of the flowing fluid to thermal energy to further heat the stream to cause pyrolysis of the methane in the feed, which may occur in the expansion section 60 and/or a downstream reaction section 65 of the reactor. The fluid is quickly quenched in a quench zone 72 to stop the pyrolysis reaction from further conversion of the desired acetylene product to other compounds. A quench injection apparatus 75 may be used to introduce a quenching fluid, for example water or steam into the quench zone 72. The quench injection apparatus 75 may include for example one or more of the following: spray bars, spray nozzles, or any other apparatus appropriate for injecting a quench fluid.

The reactor effluent exits the reactor via outlet 80 and as mentioned above forms a portion of the hydrocarbon stream. The effluent will include a larger concentration of acetylene than the feed stream and a reduced concentration of methane relative to the feed stream. The reactor effluent stream may also be referred to herein as an acetylene stream as it includes an increased concentration of acetylene. The acetylene stream may be an intermediate stream in a process to form another hydrocarbon product or it may be further processed and captured as an acetylene product stream. In one example, the reactor effluent stream has an acetylene concentration prior to the addition of quenching fluid ranging from about 2 mol-% to about 30 mol-%. In another example, the concentration of acetylene ranges from about 5 mol-% to about 25 mol-% and from about 8 mol-% to about 23 mol-% in another example.

The reactor vessel 10 includes a reactor shell 11. It should be noted that the term “reactor shell” refers to the wall or walls forming the reactor vessel, which defines the reactor chamber 15. The reactor shell 11 will typically be an annular structure defining a generally hollow central reactor chamber 15. The reactor shell 11 may include a single layer of material, a single composite structure or multiple shells with one or more shells positioned within one or more other shells. The reactor shell 11 also includes various zones, components, and or modules, as described above and further described below for the different zones, components, and or modules of the supersonic reactor 5. The reactor shell 11 may be formed as a single piece defining all of the various reactor zones and components or it may be modular, with different modules defining the different reactor zones and/or components.

By one approach, at least a portion of the reactor shell 11 is constructed of a material having a high melting temperature to withstand the high operating temperatures of the supersonic reactor 5. In one approach, one or more materials forming the portion of the reactor shell 11 may have a long low-cycle fatigue life, high yield strength, resistance to creep and stress rupture, oxidation resistance, and compatibility with coolants and fuels. In one example, at least a portion of the reactor shell 11 is formed of a material having high thermal conductivity. In this manner, heat from reactor chamber 15 may be quickly removed therefrom. This may restrict a skin temperature of an internal surface of the reactor shell 11 from being heated to temperatures at or near the reactor temperature, which may cause melting, chemical fire, or other deterioration, to the reactor shell 11 walls. In one example, the one or more portions of the reactor are formed from a material having a thermal conductivity of between about 200 and about 500 W/m-K. In another example, the thermal conductivity is between about 300 and about 450 W/m-K. In yet another example, the thermal conductivity is between about 200 and about 346 W/m-K and may be between about 325 and about 375 W/m-K in yet another example.

It has been found that according to this approach, the reactor shell may be formed from a material having a relatively low melting temperature as long as the material has a very high conductivity. Because heat from the reaction chamber 15 is quickly removed in this approach, the reactor shell 11 is not exposed to as high as the temperature. In this regard, the forming by reactor shell portion from a material having a high thermal conductivity, the material may have a melting temperature below the temperature in the reactor chamber 15. In one example, the portion of the reactor shell 11 is formed from a material having a melting temperature of between about 500 and about 2000° C. In another example, the reactor shell 11 portion may be formed from a material having a melting temperature of between about 800 and about 1300° C. and may be formed from a material having a melting temperature of between about 1000 and about 1200° C. in another example.

By one approach, the material having a high thermal conductivity includes a metal or metal alloy. In one approach, one or more portions of the reactor shell 11 may be formed from copper, silver, aluminum, zirconium, niobium, and their alloys. In this regard, it should be noted that one or more of the materials listed above may also be used to form a coating on a reactor shell substrate or to form a layer of a multilayer reactor shell 11. By one approach, the reactor shell 11 portion includes copper or a copper alloy. In one example, the reactor shell portion includes a material selected from the group consisting of copper chrome, copper chrome zinc, copper chrome niobium, copper nickel and copper nickel tungsten. In another example, the reactor shell portion comprises niobium-silver. In order to enhance the removal of heat from reactor chamber, cooling may be used to more quickly remove the heat from the reactor chamber so that a temperature thereof is maintained below and allowable temperature.

The foregoing description provides several approaches with regard to a reactor shell 11 or a portion of a reactor shell 11. In this manner, it should be understood that at least a portion of the reactor shell 11 may refer to the entire reactor shell 11 or it may refer to less than the entire reactor shell as will now be described in further detail. As such, the preceding description for ways to improve the construction and/or operation of at least a portion of the reactor shell 11 may apply generally to any portion of the reactor shell and/or may apply to the following specifically described portions of the reactor shell. Greater detail regarding the reactor 5 may be found in co-pending and commonly-assigned U.S. patent application Ser. No. 13/967,334, the contents of which are incorporated herein by reference in their entirety

In one example, the reactor effluent stream after pyrolysis in the supersonic reactor 5 has a reduced methane content relative to the methane feed stream ranging from about 15 mol-% to about 95 mol-%. In another example, the concentration of methane ranges from about 40 mol-% to about 90 mol-% and from about 45 mol-% to about 85 mol-% in another example.

The reactor effluent stream 2 generated from a feedstock may comprise particulate matter or liquid droplets, for example soot particles or excess quench fluid. Therefore, in a preferred embodiment the reactor effluent exiting a supersonic reactor 5 is contacted with scrubbing liquid in a soot scrubber/quench tower 104 to remove particulate matter, in particular soot, or liquid droplets thereby obtaining a fully cooled and scrubbed reactor effluent stream 17. The reactor effluent stream exiting the supersonic reactor 5 is generally at elevated temperature and/or elevated pressure. To avoid additional cooling and/or depressurising steps, the scrubbing step in the soot scrubber 104 is preferably performed at elevated temperature and/or at elevated pressure. The preferred operating temperature will vary depending on the quench fluid and scrubbing fluid selected. In one example, the quench fluid is water and the scrubbing liquid is water. In this case the preferred operating temperature will be below the boiling point for water, for example less than about 100° C. Preferably, the pressure at which the reactor effluent stream 2 is contacted with scrubbing liquid is less than 50 psig, more preferably less than 30 psig. Spent scrubbing liquid with the soot exits scrubber 104 via line 8.

Thereafter, an acid wash system is utilized to decompose any copper acetylides that may be present in the reactor effluent stream 2. A wide variety of acids may be used, including dilute hydrochloric acid. Dilute acids are preferred in order to minimize the possibility of metal erosion due to acid conditions. Acid may be provided by acid feed stream 9. The reactor effluent from stream 17 and the acid wash from stream 9 are brought together in a “mixer” 103. Generally speaking, mixer 103 design will be conventional with the objective of ensuring good contact between the effluent and the acid with various types of contactor applicable, for example, scrubbers, countercurrent towers. For example, in one embodiment, the acid solution can be sprayed to the reactor effluent in a tower. In another version, the reactor effluent can be bubbled through an acid pool. In yet another embodiment, a static mixer may be used to mix the reactor effluent with the acid. During this step, the conditions should be chosen so as to maintain the effluent stream in the liquid phase since this will favor removal of the copper acetylide species. In one embodiment, the acid stream 9 may be introduced with the quench fluid 7 in reactor vessel 5. In another embodiment, the mixer 103 and quench tower 104 may be combined into a single operation with the acid stream 9 introduced with the scrubbing liquid.

Following the acid wash and mixing in unit 103, the acid is neutralized and recovered in unit 106. Neutralization may be achieved by the addition of a suitable amount of basic material, such as caustic or other basic composition. Recovery of the acid may be achieved by any suitable gas/liquid separation means, such as a coalescer separator. The coalescer is provided for removing liquids, such as the neutralized acid, from the gaseous phase of hydrocarbons. Suitable coalescers to remove the neutralized acids are known in the art and are commercially available. The coalescer promotes the coalescence of the discontinuous or highly divided phase of the neutralized acid/hydrocarbon effluent mixture in the form of finely divided water droplets into larger and coarser droplets.

The coalescing unit and the separation unit may suitably be contained in a housing which provides and adequate number of coalescing/separating elements with these elements being suitably arranged inside the housing for reasons of functionality and operating convenience. A suitable arrangement is shown in U.S. Pat. No. 5,443,724, using coalescer and separator cartridge elements arranged in super posed relationship with one another in a cylindrical type housing which permits ready access to the cartridges when they require replacement. However, other configurations may be used and reference is made to commercial suppliers of this equipment including Pall Corporation of East Hills, N.Y. 1 1548.

In an embodiment, the quench fluid and the acid steam 9 may be liquids that are insoluble, for example when an oil quench is employed. In this case, the acid may be recovered using liquid-liquid separation techniques known to one skilled in the art.

As shown in FIG. 1, spent neutralized acid leaves the unit 106 via line 18. As noted above, neutralized acid may be recycled. The reactor effluent stream exits unit 106 via line 19. Due to contacting with the dilute acid, the effluent stream in line 19 includes more water than in line 17. As such, line 19 may continue downstream to a series of dryer units included with in a optional dehydration zone 112. In dehydration zone 112, excess water is removed by any suitable means known in the art. Water leaves zone 112 via line 23, and the dried effluent continues to hydrocarbon conversion zone 100.

Still referring to FIG. 2, the reactor effluent stream having a higher concentration of acetylene may be passed to a downstream hydrocarbon conversion zone 100 where the acetylene may be converted to form another hydrocarbon product. The hydrocarbon conversion zone 100 may include a hydrocarbon conversion reactor for converting the acetylene to another hydrocarbon product. Additionally, it should be understood that the hydrocarbon conversion zone 100 may include a variety of other hydrocarbon conversion processes instead of or in addition to the exemplary hydrogenation reactor mentioned herein, or a combination of hydrocarbon conversion processes. Similarly, unit operations illustrated in FIG. 2 may be modified or removed and are shown for illustrative purposes and not intended to be limiting of the processes and systems described herein. Specifically, it has been identified that several other hydrocarbon conversion processes, other than those disclosed in previous approaches, may be positioned downstream of the supersonic reactor 5, including processes to convert the acetylene into other hydrocarbons, including, but not limited to: alkenes, alkanes, methane, acrolein, acrylic acid, acrylates, acrylamide, aldehydes, polyacetylides, benzene, toluene, styrene, aniline, cyclohexanone, caprolactam, propylene, butadiene, butyne diol, butandiol, C₂-C₄ hydrocarbon compounds, ethylene glycol, diesel fuel, diacids, diols, pyrrolidines, and pyrrolidones.

By one approach, the reactor effluent stream is reacted to form another hydrocarbon compound. In this regard, the reactor effluent portion of the hydrocarbon stream may be passed from the reactor outlet to a downstream hydrocarbon conversion process for further processing of the stream. While it should be understood that the reactor effluent stream may undergo several intermediate process steps, such as, for example, water removal, adsorption, and/or absorption to provide a concentrated acetylene stream, these intermediate steps will not be described in detail herein.

It will be appreciated that the acid wash system described herein for the removal of copper acetylides need not be employed during an entirety of the operation of the supersonic reactor 5 (although it may be). For example, upon shutting down the supersonic reactor it may be beneficial to acid wash the reactor to decompose any acetylides that may have formed on the internal surfaces during operation or the transients during shutdown. To this end, an acid stream may be injected into supersonic reactor 5 using one or more of the inlets described above, alternatively the reactor may include a dedicated inlet for the acid wash. The purpose of this shut-down procedure is to ensure that there are no deposits of acetylides in the reactor vessel and downstream equipment before opening vessels for maintenance. Acid is introduced at the reactor inlet through a dedicated inlet or one of the inlets provided to supply a process stream to the reactor. As noted above, the acid wash is found to decompose copper acetylides back to acetylene. The reactor is safe to open for maintenance or otherwise exposed to the open air at the end of the gas purge step and depressurization.

By one approach, hydrocarbon conversion zone 100 may include a hydrogenation reactor. In one embodiment, said hydrogenation catalyst may contain copper. The Cu or CuO active sites present in hydrogenation catalyst is found to interact readily with acetylene leading to copper acetylide formation. Once the acetylides are dried due to gas-purge (with N₂) during shutdown, they are more susceptible to explosion. As such, some embodiments of the present disclosure employ an acid-wash process step during shut-down to solve the problem. The purpose of this shut-down procedure is to ensure that there are no deposits of acetylides in the reactor vessel and downstream equipment before opening vessels for maintenance. Acid is introduced at the reactor inlet through a dedicated inlet or one of the inlets provided to supply a process stream to the reactor. As noted above, the acid wash is found to decompose copper acetylides back to acetylene. Once the reactor shutdown is initiated, a dilute solution of acid is used to displace the process fluid from the reactor. A flow of acid is maintained at an LHSV>0.5 for a minimum period of 30 minutes to ensure that the copper acetylide has decomposed. This acid-wash step can be followed by a gas-purge step to ensure that all hydrocarbon and acid solution is removed from the reactor. The reactor is safe to be unloaded at the end of the gas purge step and depressurization.

Accordingly, apparatus and methods for converting methane in a hydrocarbon stream to acetylene using a supersonic flow reactor, while minimizing the possibility of explosion due to the presence of copper acetylides. The methane pyrolysis reactor zone presents a strong reducing environment in the presence of hydrogen and high temperature. Cu or CuO is found to interact readily with acetylene leading to copper acetylide formation. Due to the high momentum of flow in the reactor, at least a portion of the copper acetylide formed is carried in the reactor effluent, even when methods to prevent the formation of acetylides or in-situ removal procedures are used. The presently described embodiments employ a system to remove copper acetylide from the reactor effluent. The system uses an acid wash of the reactor effluent, which decomposes the acetylides. The acid wash section can be designed before or after the soot removal system, and is followed by an acid neutralization unit or acid recovery separator system.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described in an exemplary embodiment without departing from the scope, as set forth in the appended claims.

What is claimed is:

1. A system for producing acetylene from a feed stream comprising methane comprising:
a supersonic reactor for receiving the methane feed stream and heating the methane feed stream to a pyrolysis temperature;
a reactor shell of the supersonic reactor for defining a reactor chamber;
a combustion zone of the supersonic reactor for combusting a fuel source to provide a high temperature carrier gas passing through the reactor space at supersonic speeds to heat and accelerate the methane feed stream to a pyrolysis temperature;
at least a portion of the reactor shell comprises at least one of copper and a copper alloy, wherein upon heating and accelerating the methane stream, a reactor effluent is generated comprising acetylene and a copper acetylide byproduct; and
an acid washing unit that washes the reactor effluent from the supersonic reactor with an acid to decompose the copper acetylide byproduct.

2. The system of claim 1, wherein the acid washing unit comprises an acid sprayer unit.

3. The system of claim 1, wherein the acid washing unit comprises an acid pool.

4. The system of claim 1, wherein the acid washing unit comprises a static mixer.

5. The system of claim 1, further comprising a separation system for separating the acid from the reactor effluent.

6. The system of claim 1, further comprising a drier unit for removing water from the reactor effluent.

7. The system of claim 1, further comprising a hydroprocessing unit for converting the reactor effluent into an olefin product.

8. The system of claim 7, wherein the hydroprocessing unit includes a catalyst that contains copper and the hydroprocessing unit and the catalyst are acid washed upon shutdown of the supersonic reactor.

9. The system of claim 1, wherein the reactor portion has a melting temperature of between about 500 and about 2000° C., and wherein the reactor portion material has a thermal conductivity of between about 300 and about 450 W/m-K.

10. The system of claim 1, wherein the acid is a dilute hydrochloric acid.

11. A method for producing acetylene from a feed stream comprising methane comprising:
receiving the methane feed stream and heating the methane feed stream to a pyrolysis temperature in a pyrolysis reactor, wherein at least a portion of the pyrolysis reactor comprises at least one of copper and a copper alloy;
combusting a fuel source to provide a high temperature carrier gas passing through the reactor space at supersonic speeds to heat and accelerate the methane feed stream to a pyrolysis temperature, wherein upon heating and accelerating the methane stream, a reactor effluent is generated comprising acetylene and a copper acetylide byproduct; and
washing the reactor effluent from the supersonic reactor in an acid wash to decompose the copper acetylide byproduct.

12. The method of claim 11, further comprising separating the acid from the reactor effluent.

13. The method of claim 12, wherein separating comprises passing the reactor effluent and acid through a coalescer unit.

14. The method of claim 11, further comprising removing water from the reactor effluent.

15. The method of claim 11, further comprising converting the reactor effluent into an olefin product.

16. The method of claim 11, wherein washing the reactor effluent in an acid wash comprises spraying the acid wash into the reactor effluent using an acid sprayer unit, passing the reactor effluent through an acid pool, or combining the reactor effluent and the acid wash in a static mixer.

17. The method of claim 11, wherein washing the reactor effluent in an acid wash comprises washing the reactor effluent with dilute hydrochloric acid.

18. A system for producing acetylene from a feed stream comprising methane comprising:
a supersonic reactor for receiving the methane feed stream and heating the methane feed stream to a pyrolysis temperature;
a reactor shell of the supersonic reactor for defining a reactor chamber;
a combustion zone of the supersonic reactor for combusting a fuel source to provide a high temperature carrier gas passing through the reactor space at supersonic speeds to heat and accelerate the methane feed stream to a pyrolysis temperature;
at least a portion of the reactor shell comprises at least one of copper and a copper alloy, wherein upon heating and accelerating the methane stream, a reactor byproduct is generated within the combustion zone comprising copper acetylide; and
an acid washing unit that washes the combustion zone of the supersonic reactor with an acid after shutdown of the supersonic reactor to decompose any copper acetylide byproduct that may remain in the supersonic reactor after shutdown.

19. The system of claim 18, wherein the acid wash unit comprises a dedicated inlet or an inlet provided to supply a process stream to the supersonic reactor.

20. The system of claim 18, wherein the acid is a dilute hydrochloric acid.
